(19) United States
(12) Patent Application Publication
(10) Pub. No.: US 2026/0139269 A1
(43) Pub. Date: May 21, 2026

(54) MATRIX ATTACHMENT REGIONS AND USES IN PROMOTING GENE EXPRESSION

(71) Applicant: Celltheon Corporation, Union City, CA (US)

(72) Inventor: Nikhil Goel, Union City, CA (US)

(21) Appl. No.: 19/450,815

(22) Filed: Jan. 16, 2026

Related U.S. Application Data
(62) Division of application No. 17/311,306, filed on Jun. 4, 2021, now Pat. No. 12,565,663.
(60) Provisional application No. 62/776,643, filed on Dec. 7, 2018.

(51) Int. Cl. C12N 15/85

(52) U.S. Cl. CPC C12N 15/85; C12N 2830/46

(57) ABSTRACT

The present disclosure relates to new identified matrix attachment regions (MAR) and chimeric sequences. Also disclosed are nucleotide constructs containing a MAR at a suitable location relative to a protein-coding sequence for optimal expression of the protein.

### CROSS-REFERENCE TO RELATED APPLICATIONS

This application is a divisional application of U.S. patent application No. 17/311,306 filed on June 4, 2021, which is a National Stage Application under 35 U.S.C. 371 of International Application No. PCT/US2019/064910 filed December 6, 2019, which claims the benefit under 35 U.S.C. § 119(e) of United States Provisional Application Serial Number 62/776,643 filed December 7, 2018. The contents of the aforementioned applications are incorporated by reference herein in their entirety.

### REFERENCE TO SEQUENCE LISTING

This application includes a Sequence Listing filed electronically as an XML file named "SeqKHUR25011.xml", created on January 11, 2026, with a size of 83,308 bytes. The Sequence Listing is incorporated herein by reference.

### BACKGROUND

Transcription of gene sequences (i.e., production of mRNA) is controlled at a number of different levels. Transcription initiation sites, or promoters, have different strengths, and the frequency of initiation of transcription of a given gene can also be augmented by enhancer sequences. Pausing during transcription can influence the rate of transcription and, hence, the amount of transcript produced in a given time period. Rates of pre-mRNA splicing, polyadenylation and cleavage can also influence the level of mRNA produced by a transcription unit. In addition, sequences within a mRNA molecule can regulate its transport from the nucleus to the cytoplasm, and its rate of turnover (i.e., its cytoplasmic stability).

Expression of polypeptides (e.g., therapeutic antibodies, growth factors) in vitro is important for the pharmaceutical industry, and methods to maximize protein expression are needed.

### SUMMARY

The present disclosure describes a new technology for evaluating the strength of matrix attachment regions (MAR) and shows that the technology is useful for identifying new MAR sequences that were previously unknown. Chimeric MAR sequences are also described.

The present disclosure also describes nucleotide constructs that include cis MAR placements and related other sequences for optimal expression of genes of interest (GOI).

In accordance with one embodiment of the present disclosure, therefore, provided is a recombinant polynucleotide comprising a coding sequence, a promoter configured to initiate the transcription of the coding sequence, and a matrix attachment region (MAR) core selected from the group consisting of SEQ ID NO: 1, 5, 9 and 13, and nucleic acid sequences having at least 75% sequence identity to any one of SEQ ID NO: 1, 5, 9 and 13, wherein the MAR core is capable to attach to a mammalian nuclear matrix.

Another embodiment provides a method of transfecting to a cell a coding sequence, comprising contacting the cell with a first polynucleotide comprising the coding sequence and a promoter for initiating transcription of the coding sequence, and a second, unlinked polynucleotide comprising a matrix attachment region (MAR) core selected from SEQ ID NO: 1, 5, 9 and 13, and nucleic acid sequences having at least 75% sequence identity to any one of SEQ ID NO: 1, 5, 9 and 13, wherein the MAR core is capable to attach to a mammalian nuclear matrix, under conditions for the first and second polynucleotides to transfect into the cell.

Chimeric matrix attachment regions (MAR) are also provided, comprising (a) a MAR core selected from SEQ ID NO: 1, 5, 9 and 13, and nucleic acid sequences having at least 75% sequence identity to any one of SEQ ID NO: 1, 5, 9 and 13, wherein the MAR core is capable to attach to a mammalian nuclear matrix, (b) a 5’ flanking region selected from SEQ ID NO: 2, 6, 10, 14, 18, 22, 26, 30 and 34 and nucleic acid sequences having at least 75% sequence identity to any one of

SEQ ID NO: 2, 6, 10, 14, 18, 22, 26, 30 and 34, and (c) a 3’ flanking region selected from SEQ ID NO: 3, 7, 11, 15, 19, 23, 27, 31 and 35 and nucleic acid sequences having at least 75% sequence identity to any one of SEQ ID NO: 3, 7, 11, 15, 19, 23, 27, 31 and 35, wherein the MAR core, the 5’ flanking region and the 3’ flanking region are not from the same natural MAR.

### BRIEF DESCRIPTION OF THE DRAWINGS

FIG. 1 compares the strength of two MAR sequences, MAR X29 a known MAR as control, and MAR Zebrafish, which was identified by the present technology.

FIG. 2 demonstrates the strength of the MAR Zebrafish core using as control a construct that does not contain any MAR sequences.

FIG. 3 is the vector map of a MAR core-containing construct described in Example 2.

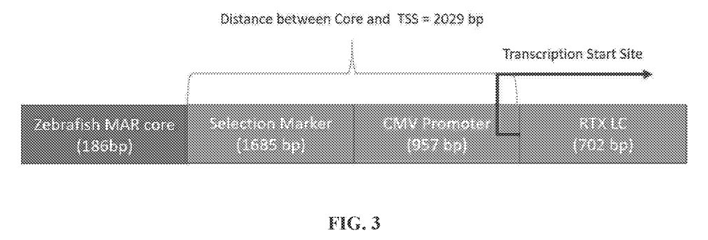

FIG. 4 is the vector map of the control construct that did not contain the MAR core element, as described in Example 2.

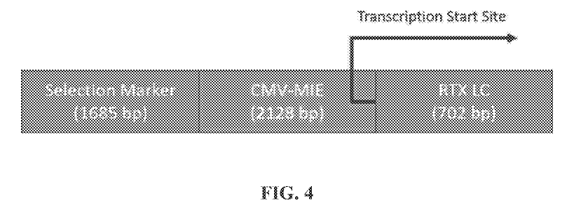

### DETAILED DESCRIPTION

I. Definitions

All numerical designations, e.g., pH, temperature, time, concentration, and molecular weight, including ranges, are approximations which are varied ( + ) or ( - ) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polynucleotide” includes a plurality of polynucleotides, including mixtures thereof.

The terms “polynucleotide” and “oligonucleotide” are used interchangeably and refer to a polymeric form of nucleotides of any length, either deoxyribonucleotides or ribonucleotides or analogs thereof. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

II. New Matrix Attachment Region (MAR) Sequences

The present disclosure describes a method to quantify the strength of a MAR element in silico and used this method to quantify the strength of new MAR sequences identified from sequence databases.

Through sequence comparison and structure-activity analyses, the instant inventors discovered that a MAR element is likely comprised of a central AT -rich core region, along with 5' and 3' flanking regions containing transcription factor binding motifs.

The core region may be enriched in the (ATAT)n microsatellite. The binding motifs may target the SATB1, NMP4, CEBP, Fast and Hox transcription factors. Additional nuclear matrix proteins that the MAR may bind to include the ARBP protein (attachment region binding protein), which recognizes the consensus sequence ATTTCAC/GTTGTAAAA in the MAR, the NMP-2 protein, localized exclusively in the nuclear matrix, the Spl, ATF, CCAAT, C/EBP, and AP-1 transcription factors, the yeast ACBP protein (ARS consensus binding protein), which interacts with the ARS element, the tissue-specific human SATB1 protein, expressed predominantly in thymus that binds to the minor groove of a special class of AT -rich MARs with A, T, or C but not G on one strand, the matrin 3 protein, an acidic protein of the internal nuclear matrix network of human and rat cells, matrin F/G, the transcription protein factor RFP.

The analytical results assisted the inventors in the search for new MAR sequences, leading to discovery of four new MAR sequences that are highly effective in promoting gene expression, as compared to a positive reference, MAR X29 (Arope et al. (2013) PLoS ONE 8(11): e79262).

It is further demonstrated that the cores of these newly identified MAR sequences can form functional MAR with the 5’ and 3’ flanking regions from other MARs, providing additional useful chimeric MAR sequences.

In accordance with one embodiment of the present disclosure, therefore, provided is a recombinant polynucleotide comprising a coding sequence, a promoter configured to initiate the transcription of the coding sequence, and a matrix attachment region (MAR) core described herein and variants thereof. Examples of such new MAR cores are listed in Table 1. See, e.g., SEQ ID NO: 1, 5, 9 and 13.

Matrix attachment regions, or MARs, are sequences in the DNA of eukaryotic chromosomes where the nuclear matrix attaches. As architectural DNA components that organize the genome of eukaryotes into functional units within the cell nucleus, MARs can mediate structural organization of the chromatin within the nucleus. These elements constitute anchor points of the DNA for the chromatin scaffold and serve to organize the chromatin into structural domains. The dynamic and complex organization of the chromatin mediated by MAR elements plays an important role in the regulation of gene expression.

Variants of the MAR cores are nuclei acid sequences that have certain sequence identity (e.g., at least 75%, 80%, 85%, 90%, 95%, 97%, 98%, or 99%) to the reference MAR core (e.g., SEQ ID NO: 1, 5, 9 and 13) and have the expected MAR function (e.g., capability to attach to a mammalian nuclear matrix).

In some embodiments, the variants of the MAR cores are AT-rich, e.g., having at least 75%, 80%, 85%, 90%, or 95% A or T in the sequence.

The MAR core can be present along with a 5’ flanking region of a MAR, such as any one of SEQ ID NO: 2, 6, 10, 14 and 18 or their variants (an nucleic acid sequence having at least 75%, 80%, 85%, 90%, 95%, 97%, 98%, or 99% sequence identity to any one of SEQ ID NO: 2, 6, 10, 14 and 18). In some embodiments, the 5’ flanking region is 5’ to, and within 100 nucleotides from, the MAR core.

In some embodiments, the MAR core can be present further along with a 3’ flanking region of a MAR, such as any one of SEQ ID NO: 3, 7, 11, 15 and 19 or their variants (an nucleic acid sequence having at least 75%, 80%, 85%, 90%, 95%, 97%, 98%, or 99% sequence identity to any one of SEQ ID NO: 3, 7, 11, 15 and 19). In some embodiments, the 3’ flanking region is 3’ to, and within 100 nucleotides from, the MAR core.

Non-limiting MAR sequences that include a MAR core, a 5’ flanking region and a 3’ flanking region include SEQ ID NO: 4, 8, 12 and 16, and nucleic acid sequences having at least 75%, 80%, 85%, 90%, 95%, 97%, 98%, or 99% sequence identity to any one of SEQ ID NO: 4, 8, 12 and 16, wherein the MAR is capable to attach to a mammalian nuclear matrix.

III. Chimeric MAR Sequences

As provided, the cores of these newly identified MAR sequences can form functional MAR with the 5’ and 3’ flanking regions from other MARs, providing additional useful chimeric MAR sequences.

In one embodiment, a chimeric MAR of the present disclosure includes a MAR core as disclosed herein with a 5’ flanking region on the 5’ side. In one embodiment, a chimeric MAR of the present disclosure includes a MAR core as disclosed herein with a 3’ flanking region on the 3’ side. In one embodiment, a chimeric MAR of the present disclosure includes a MAR core as disclosed herein with a 5’ flanking region on the 5’ side and a 3’ flanking region on the 3’ side.

The 5’ flanking region can be selected from any 5’ flanking region or 3’ flanking region known in the art or disclosed herein. In some embodiments, the 5’ flanking region can be selected from any 5’ flanking region known in the art or disclosed herein.

The 3’ flanking region can be selected from any 5’ flanking region or 3’ flanking region known in the art or disclosed herein. In some embodiments, the 5’ flanking region can be selected from any 3’ flanking region known in the art or disclosed herein.

Each of the MAR core, MAR 5’ flanking region and MAR 3’ flanking region can be substituted with a nucleic acid variant (e.g., having at least 75%, 80%, 85%, 90%, 95%, 97%, 98%, or 99% sequence identity to the reference sequence) as well.

The MAR cores disclosed herein include SEQ ID NO: 1, 5, 9 and 13. The 5’ flanking regions disclosed herein include SEQ ID NO: 2, 6, 10, 14, 18, 22, 26, 30 and 34. The 3’ flanking regions disclosed herein include SEQ ID NO: 3, 7, 11, 15, 19, 23, 27, 31 and 35.

The MAR flanking sequences newly identified here can also be fused to the other core sequences, either newly discovered here or known, to generate new chimeric MAR sequences, in some embodiment. In some embodiments, a chimeric MAR sequence includes any one of the MAR core regions of Table 1, any one of the MAR 5’ flanking sequences of Table 1, and any one of the MAR 3’ flanking sequences of Table 1.

IV. Gene Expression

The MAR sequences and constructs disclosed herein can be useful for promoting gene expression in cells. When a gene of interest is included in a construct that contains the MAR (MAR alone or with 5’ and/or 3’ flanking regions), the construct can be introduced into a host cell for expressing the gene of interest.

The MAR sequences disclosed herein can also be used in trans to promote gene expression, as a trans-acting element. For instance, a construct that includes any of the MAR sequences disclosed herein (MAR alone or with 5’ and/or 3’ flanking regions, wild-type or chimeric) can be introduced into a cell that is further transfected (or has been transfected) with a construct containing a gene of interest (GOI, or transgene) such that the MAR sequence can assist the expression of the GOI.

In accordance with one embodiment of the present disclosure, therefore, provided is a method of transfecting to a cell a coding sequence, comprising contacting the cell with a first polynucleotide comprising the coding sequence and a promoter for initiating transcription of the coding sequence, and a second, unlinked polynucleotide comprising a matrix attachment region (MAR) core as disclosed herein, under conditions for the first and second polynucleotides to transfect into the cell. In some embodiments, the MAR core is selected from SEQ ID NO: 1, 5, 9 and 13, and nucleic acid sequences having at least 75% sequence identity to any one of SEQ ID NO: 1, 5, 9 and 13, wherein the MAR core is capable to attach to a mammalian nuclear matrix,

In some embodiments, the second polynucleotide further comprises a 5’ flanking region selected from SEQ ID NO: 2, 6, 10, 14 and 18 and nucleic acid sequences having at least 75% sequence identity to any one of SEQ ID NO: 2, 6, 10, 14 and 18. In some embodiments, the 5’ flanking region is 5’ to, and within 100 nucleotides from, the MAR core.

In some embodiments, the second polynucleotide further comprises a 3’ flanking region selected from SEQ ID NO: 3, 7, 11, 15 and 19 and nucleic acid sequences having at least 75% sequence identity to any one of SEQ ID NO: 3, 7, 11, 15 and 19. In some embodiments, the 3’ flanking region is 3’ to, and within 100 nucleotides from, the MAR core.

In some embodiments, the entire MAR sequence is selected from SEQ ID NO: 4, 8, 12 and 16, and nucleic acid sequences having at least 75% sequence identity to any one of SEQ ID NO: 4, 8, 12 and 16, wherein the MAR is capable to attach to a mammalian nuclear matrix.

Also provided are transfected cell prepared by the methods disclosed herein.

In any of the constructs or vectors disclosed herein, additional transcriptional regulatory sequences and/or post-transcriptional regulatory sequences can be included. Transcriptional regulatory sequences can include, for example, promoters, enhancers and polyadenylation signals. Post-transcriptional regulatory sequences include, for example, introns and PREs.

In certain embodiments, a multiple cloning site (MCS), also known as a “polylinker,” is present in the vector to facilitate insertion of heterologous sequences. For example, a MCS can be disposed between a promoter and a polyadenylation signal, to facilitate insertion of transgene sequences. In vectors containing transgene sequences, the portion of the vector containing a promoter, transgene sequences a polyadenylation signal is denoted the “expression cassette.”

Promoters active in eukaryotic cells are known in the art. Exemplary eukaryotic promoters include, for example SV40 early promoter, SV40 late promoter, cytomegalovirus (CMV) promoter, cytomegalovirus major immediate early (CMV-MIE) promoter, EF1 -alpha (translation elongation factor- 1 a subunit) promoter, Ubc (ubiquitin C) promoter, PGK (phosphogly cerate kinase) promoter, actin promoter and others. See also Boshart et al., GenBank Accession No.K03104; Uetsuki et al. (1989) J. Biol. Chem. 264:5791-5798; Schorpp et al. (1996) Nucleic Acids Res. 24: 1787-1788; Hamaguchi et al. (2000) J. Virology 74: 10778-10784; Dreos et al. (2013) Nucleic Acids Res. 41(D1):D157-D164 and the eukaryotic promoter database, accessed on July 16, 2014.

Enhancers can also be included on the vector. Non-limiting examples include those in CMV promoter and intron A sequences. Five embryonic stem cell (ESC) transcription factors were previously shown to occupy super-enhancers (Oct4, Sox2, Nanog, Klf4, and Esrrb), and there are many additional transcription factors that contribute to the control of ESCs. Six additional transcription factors (Nr5a2, Prdml4, Tcfcp211, Smad3, Stat3, and Tcf3) occupy both typical enhancers and super-enhancers and that all of these are enriched in super-enhancers. Any of these or further known in the art can be used herein.

Polyadenylation signals that are active in eukaryotic cells are known in the art and include, but are not limited to, the SV40 polyadenylation signal, the bovine growth hormone (BGH) polyadenylation signal and the herpes simplex virus thymidine kinase gene polyadenylation signal. The polyadenylation signal directs 3' end cleavage of pre-mRNA, polyadenylation of the pre-mRNA at the cleavage site and termination of transcription downstream of the polyadenylation signal. A core sequence AAUAAA is generally present in the polyadenylation signal. See also Cole et al. (1985) Mol. Cell. Biol. 5:2104-2113.

Exemplary introns that can be used in the vectors disclosed herein include the b-globin intron and the first intron of the human/ mouse/ rat / other species cytomegalovirus major immediate early (MIE) gene, also known as “intron A.”

Additional post-transcriptional regulatory elements that can be included in the vectors of the present disclosure include, without limitation, the 5'-untranslated region of CMV MIE, the human Hsp70 gene, the SP163 sequence from the vascular endothelial growth factor (VEGF) gene, and the tripartite leader sequence associated with adenovirus late mRNAs. See, for example, Mariati et al. (2010) Protein Expression and Purification 69:9-15.

In certain embodiments, the vectors disclosed herein contain nucleotide sequences encoding a selection marker that functions in eukaryotic cells (i.e., a eukaryotic selection marker), such that when appropriate selection is applied, cells that do not contain the selection marker die or grow appreciably more slowly that do cells that contain the selection marker. An exemplary selection marker that functions in eukaryotic cells is the glutamine synthetase (GS) gene; selection is applied by culturing cells in medium lacking glutamine or selection with L-Methioniene Sulfoximine or both. Another exemplary selection marker that functions in eukaryotic cells is the gene encoding resistance to neomycin (neo); selection is applied by culturing cells in medium containing neomycin, Geneticine or G418. Additional selection markers include dihydrofolate reductase (DHFR, imparts resistance to methotrexate), puromycin-N-acetyl transferase (provides resistance to puromycin) and hygromycin kinase (provides resistance to hygromycin B). Yet additional selection markers that function in eukaryotic cells are known in the art.

The sequences encoding the selection marker(s) described above are operatively linked to a promoter and a polyadenylation signal. As stated above, promoters and polyadenylation signals that function in eukaryotic cells are known in the art.

In certain embodiments, a vector as disclosed herein can contain two or more expression cassettes. For example, a vector containing two expression cassettes, one of which encodes an antibody heavy chain, and the other of which encodes an antibody light chain can be used for production of functional antibody molecules.

The vectors disclosed herein also contain a replication origin that functions in prokaryotic cells (i.e., a prokaryotic replication origin). Replication origins that functions in prokaryotic cells are known in the art and include, but are not limited to, the oriC origin of E. coir, plasmid origins such as, for example, the pSClOl origin, the pBR322 origin (rep) and the pUC origin; and viral (i.e., bacteriophage) replication origins. Methods for identifying procaryotic replication origins are provided, for example, in Sernova & Gelfand (2008) Brief. Bioinformatics 9(5):376-391.

The vectors disclosed herein also contain a selection marker that functions in prokaryotic cells (i.e., a prokaryotic selection marker). Selection markers that function in prokaryotic cells are known in the art and include, for example, sequences that encode polypeptides conferring resistance to any one of ampicillin, kanamycin, chloramphenicol, or tetracycline. An example of a polypeptide conferring resistance to ampicillin (and other beta-lactam antibiotics) is the beta-lactamase (bla) enzyme. Kanamycin resistance can result from activity of the neomycin phosphotransferase gene; and chloramphenicol resistance is mediated by chloramphenicol acetyl transferase.

Exemplary transgenes include any recombinant protein or e.g., hormones (such as, for example, growth hormone) erythropoietin, antibodies, polyclonal, monoclonal antibodies (e.g, rituximab), antibody conjugates, fusion proteins (e.g, IgG-fusion proteins), interleukins, CD proteins, MHC proteins, enzymes and clotting factors. Antibody heavy chains and antibody light chains can be expressed from separate vectors, or from the same vector containing two expression cassettes.

The present disclosure provides methods for expressing a recombinant polypeptide in a cell. The methods comprise introducing a vector as described herein into a cell and culturing the cell under conditions in which the vector is either transiently or stably maintained in the cell. Cells can be prokaryotic or eukaryotic, such as stable cell lines generated by targeted integration with CRISP/Cas9. Cultured eukaryotic cells, that can be used for expression of recombinant polypeptides, are known in the art. Such cells include fungal cells ( e.g ., yeast), insect cells, plant cells and mammalian cells. Accordingly, the present disclosure provides a cell comprising a vector as described herein.

Exemplary yeast cells include, but are not limited to, Trichoderma sp., Pichia pastoris, Schizosaccharomyces pombae and Saccharomyces cerevisiae. Exemplary insect cell lines include, but are not limited to, Sf9, Sf21, and Drosophila S2 cells. Exemplary plant cells include, but are not limited to, Arabidopsis cells and tobacco BY2 cells.

Cultured mammalian cell lines, useful for expression of recombinant polypeptides, include Chinese hamster ovary (CHO) cells, human embryonic kidney (HEK) cells, virally transformed HEK cells (e.g., HEK293 cells), NS0 cells, SP20 cells, CV-1 cells, baby hamster kidney (BHK) cells, 3T3 cells, Jurkat cells, HeLa cells, COS cells, PERC.6 cells, CAP® cells and CAP-T® cells (the latter two cell lines being commercially available from Cevec

Pharmaceuticals, Cologne, Germany). A number of derivatives of CHO cells are also available such as, for example, CHO-DXB11, CHO-DG-44, CHO-K1, CHO-S, or engineered CHO cells such as CHO-M, CK1 SV CHO, and CHOZN. Mammalian primary cells can also be used.

In certain embodiments, the cells are cultured in a serum-free medium. For example, for manufacture of therapeutic proteins for administration to patients, expressing cells must be grown in serum-free medium. In additional embodiments, the cells have been pre-adapted for growth in serum-free medium prior to being used for polypeptide expression.

The vectors as described herein can be introduced into any of the aforementioned cells using methods that are known in the art. Such methods include, but are not limited to, polyethylene glycol (PEG)-mediated methods, electroporation, biolistic delivery (z.e., particle bombardment), protoplast fusion, DEAE-dextran-mediated methods, and calcium phosphate co precipitation. See also, Sambrook et al. “Molecular Cloning: A Laboratory Manual,” Third Edition, Cold Spring Harbor Laboratory Press, 2001; and Ausubel et al. ,“Current Protocols in Molecular Biology,” John Wiley & Sons, New York, 1987 and periodic updates.

Standard methods for cell culture are known in the art. See, for example, R. I. Freshney “Culture of Animal Cells: A Manual of Basic Technique,” Fifth Edition, Wiley, New York, 2005.

### EXAMPLES

The disclosure is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

Example 1. Search for New MAR

This example developed a method to quantify the strength of a matrix attachment region (MAR) element in silico and used this method to quantify the strength of a few exemplary MAR sequences.

Motif-Based MAR Identification

A literature search was performed to identify all motifs related to MAR identification, specifically looking for MAR specific transcription factors, chromatin binding domains, DNA bending and DNA unwinding sequences. Potentially useful characteristics were summarized and used for further MAR evaluation.

A MAR element is likely comprised of a central AT-rich core region and the 5' and 3' flanking regions containing transcription factor binding motifs. The core region may be enriched in the (ATAT)n microsatellite.

A MAR may contain and be generally categorized by the inclusion of a“MAR recognition signature,” a bipartite element composed of two distinct sequences AATAAYAA and AWWRTAANNWWGNNNC. Other MAR indicative sequences consist of various transcription factors and DNA structure motifs. Exemplary sequences include the DNA-unwinding motif, AP-1, A-box and T-box, NMP-2, SATB1, Hox4D, TEF, Pitl and Fast.

Four new MAR elements were identified through the search, named as MAR Zebrafish, MAR Cyprinus Carpio, MAR 12-RP13, and MAR 17XX_fos, respectively. Their, as well as a few known ones’ (MAR 1-68, MAR S4, MAR X29, Mouse c-myc 5MAR, and SPR2A-MAR), completed sequences, core and 5’ and 3’ flanking regions of the core are listed in Table 1.

Table 1. MAR Sequences

underlined.

Example 2. Testing of MAR Zebrafish

Experimental testing of one of the identified MAR elements, MAR Zebrafish, was conducted in comparison to the strongest known MAR from literature and was shown to be two times more potent.

In vitro comparison was made between MAR Zebrafish, a newly identified MAR element, and MAR X29 (reference, see Arope et al. (2013) PLoS ONE 8(11): e79262).

Experimental testing of the MAR Zebrafish core, which did not include the flanking regions of the full-length MAR Zebrafish, was then conducted in comparison to a construct that did not contain any MAR sequences.

### Methods and materials

Neon transfection was done with CT MCB cells. BalanCD + 4mM Gin Media was used for transfection. Transient Transfection done by Bio-Rad: Gene Pulser II setting at 900uF, 300v resistance 0. Cells were split before 24 hours of transfection. Day 0 viability & VCD are measured as million cells/ml. Transfections were done using 5pg of Heavy Chain & Light chain DNA. 10pg of GFP DNA was used as an external control to check the transfection efficiency. Stable pools were generated by maintaining methionine sulfoximine (MSX) at levels resulting in selective pressure that favored the growth of cells expressing the recombinant protein of interest, Rituximab Light Chain, for 14 days. Resulting recombinant pools were compared in 14 day fed batch quantitation assays. Titers at the day of harvest and throughout culture were analyzed by biolayer interferometry or ELISA. The experimental method above for pool generation was repeated to generate the pools for testing the MAR Zebrafish core, using only one concentration of MSX, 80 mM.

### Results

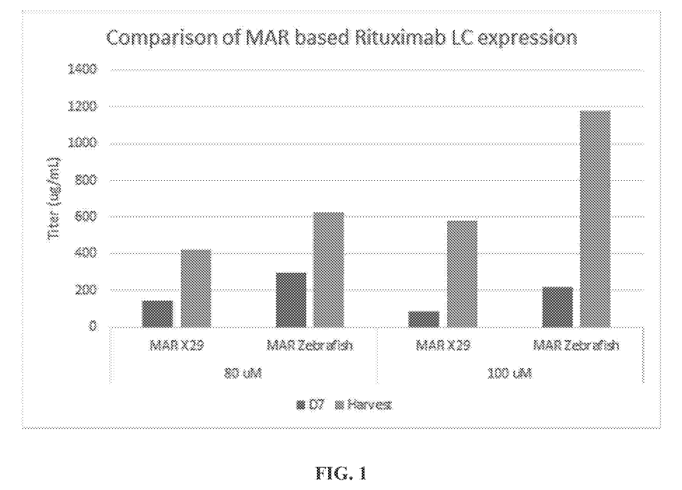

The results of the in vitro comparison of MAR X29 and MAR Zebrafish are shown in FIG. 1. At both selection marker concentrations (methionine sulfoximine (MSX) at 80mM and 100 mM), MAR Zebrafish outperformed MAR X29 by a considerable margin. This example, therefore, validated the search and in silico approach developed in Example 1.

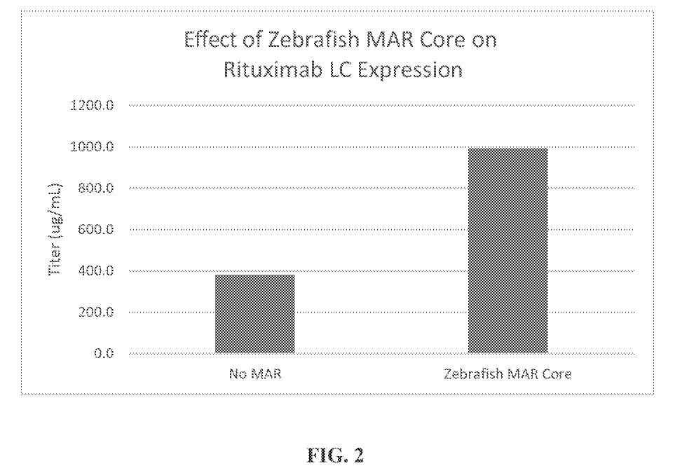

In a further experiment, the potency of the MAR core sequence alone was tested in comparison to a construct without any MAR elements. As shown in FIG. 2, The MAR Zebrafish core sequence resulted in significantly higher titers than the no MAR control.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A method of transfecting to a cell a coding sequence, comprising contacting the cell with a first polynucleotide comprising the coding sequence and a promoter for initiating transcription of the coding sequence, and a second, unlinked polynucleotide comprising a matrix attachment region (MAR) core selected from SEQ ID NO: 1, 5, 9 and 13, and nucleic acid sequences having at least 90% sequence identity to any one of SEQ ID NO: 1, 5, 9 and 13, wherein the MAR core is capable to attach to a mammalian nuclear matrix, under conditions for the first and second polynucleotides to transfect into the cell.

2. The method of claim 1, wherein the second polynucleotide further comprises a 5’ flanking region selected from SEQ ID NO: 2, 6, 10, and 14 and nucleic acid sequences having at least 75% sequence identity to any one of SEQ ID NO: 2, 6, 10, and 14.

3. The method of claim 2, wherein the 5’ flanking region is 5’ to, and within 100 nucleotides from, the MAR core.

4. The method of claim 1, wherein the second polynucleotide further comprises a 3’ flanking region selected from SEQ ID NO: 3, 7, 11, and 15 and nucleic acid sequences having at least 75% sequence identity to any one of SEQ ID NO: 3, 7, 11, and 15.

5. The method of claim 4, wherein the 3’ flanking region is 3’ to, and within 100 nucleotides from, the MAR core.

6. The method of claim 1, wherein the second polynucleotide further comprises a MAR selected from SEQ ID NO: 4, 8, 4-8., and nucleic acid sequences having at least 75% sequence identity to any one of SEQ ID NO: 4, 8, 4-8., wherein the MAR is capable to attach to a mammalian nuclear matrix.

7. A transfected cell prepared by the method of claim 1.

8. A chimeric matrix attachment region (MAR), comprising:
(a) a MAR core selected from SEQ ID NO: 1, 5, 9 and 13, and nucleic acid sequences having at least 75% sequence identity to any one of SEQ ID NO: 1, 5, 9 and 13, wherein the MAR core is capable to attach to a mammalian nuclear matrix,
(b) a 5’ flanking region selected from SEQ ID NO: 2, 6, 10, 14, 18, 22, 26, 30 and 34 and nucleic acid sequences having at least 75% sequence identity to any one of SEQ ID NO: 2, 6, 10, 14, 18, 22, 26, 30and 34, and
(c) a 3’ flanking region selected from SEQ ID NO: 3, 7, 11, 15, 19, 23, 27, 31 and 35 and nucleic acid sequences having at least 75% sequence identity to any one of SEQ ID NO: 3, 7, 11, 15, 19, 23, 27, 31 and 35, wherein the MAR core, the 5’ flanking region and the 3’ flanking region are not from the same natural MAR.
